(19) United States
(12) Patent Application Publication
(10) Pub. No.: US 2020/0345562 A1
(43) Pub. Date: Nov. 5, 2020

(54) Hygiene product in underpants form

(71) Applicant: W. Pelz GmbH & Co. KG, Wahlstedt (DE)

(72) Inventor: Andreas Bark, Wahlstedt (DE)

(73) Assignee: W. Pelz GmbH & Co. KG, Wahlstedt (DE)

(21) Appl. No.: 16/861,738

(22) Filed: Apr. 29, 2020

(30) Foreign Application Priority Data
Apr. 30, 2019 (EP) 19 172 060.6

(51) Int. Cl. A61F 13/513; A61F 13/496; A61F 13/84; A61F 13/15

(52) U.S. Cl. CPC A61F 13/51394; A61F 13/496; A61F 13/84; A61F 2013/1591; A61F 2013/15869; A61F 2013/15967; A61F 13/15577

(57) ABSTRACT

A hygiene product in underpants form comprising a front part, a rear part, and a crotch region connecting the front part to the rear part, the front and rear parts defining a hip opening at the upper edge, the front and rear parts and crotch region defining leg openings at two lateral edges, an absorbent core being arranged in the crotch region, and the front and rear parts comprising elastication means extending in parallel with the upper edge, causing the upper edge to bunch up when slack, wherein a label is attached near the elastication means on at least one side of the rear and/or front parts, the label being connected at two lateral edges, which extend perpendicularly to the upper edge, to the rear and/or front parts, the label lying flat against the side of the rear and/or front parts in the tightened state.

### CROSS-REFERENCE TO RELATED APPLICATIONS

This application claims the benefit of European Patent Application Number 19 172 060.6 filed on Apr. 30, 2019, the entire contents of which being hereby incorporated by reference in its entirety.

### FIELD OF THE INVENTION

The invention relates to a hygiene product in underpants form.

### BACKGROUND

Hygiene products in underpants form serve to collect excretions of the human body, such as urine and feces. They are used, in particular, by people who suffer from incontinence, predominantly the elderly. Underpants of this kind are manufactured from fiber materials, in particular from fiber materials containing cellulose and/or superabsorbents, and from plastics films. Such products are disposables and are disposed of after being used once. The manufacturers aim to make the properties of such underpants similar to those of conventional underwear in order to increase wearing comfort and acceptance.

In principle, hygiene products in underpants form comprise a front part, a rear part and a crotch region that connects the front part to the rear part, the front part and rear part defining a hip opening at the upper edge and the front part, rear part and crotch region defining leg openings at two lateral edges, an absorbent core being arranged in the crotch region and the front part and rear part comprising elastication means that extend in parallel with the upper edges and that cause the upper edge to bunch up when slack.

The elastication means are elastic threads (e.g. spandex threads) that are applied to the parts of the hygiene product when taut such that they cause said parts to bunch up when slack. Elastication means extending in parallel with the upper edge serve to secure the hygiene product in the hip region. Preferably, elastication means are also provided on the leg openings such that they fit tightly against the legs of the wearer.

In order to securely fasten the hygiene product against the body of the wearer, the elastication means are designed to be much stronger than the rubber bands in conventional underwear, such that the hygiene products are accordingly bunched up to a greater extent, in particular at the hip opening, in the slack state.

Hygiene products in underpants form are offered by the manufacturers in various sizes, styles and fits. In particular, there are special designs for women and for men. It is therefore necessary to provide the underpants with markings that specify the relevant design.

It is known to print the markings onto the underpants. Preferably, the imprint is made close to the upper edge on the inside of the rear part, in a similar manner to laundry labels of conventional underwear. Since the imprint is made when the rear part is lying flat, when the upper edge is bunched up, said imprint is often impossible or difficult to make out. As a result, the upper edge has to be pulled tight for clear identification of the marking. In addition, the printed marking reinforces the appearance of a disposable product.

All U.S. patents and applications and all other published documents mentioned anywhere in this application are incorporated herein by reference in their entireties. Applicant incorporates by reference herein in their entireties, patent application numbers WO 2010/050853 A1, US 2012/043244 A1, and US 2007/265591 A1.

### General Description of the Invention

Proceeding from this, the object of the invention is to provide a hygiene product in underpants form having a marking that is easier to identify and that is less reminiscent of a disposable product.

The hygiene product in underpants form according to the invention comprises a front part and a rear part as well as a crotch region connecting the front part to the rear part, the front part and rear part defining a hip opening at the upper edge and the front part, rear part and crotch region defining leg openings at two lateral edges, an absorbent core being arranged in the crotch region and the front part and rear part comprising elastication means that extend in parallel with the upper edge and that cause the upper edge to bunch up when slack, characterized in that a label is arranged in the region of the elastication means on at least one side of the rear part and/or front part, which label is only connected at the two lateral edges thereof, which extend perpendicularly to the upper edge, to the rear part and/or front part, the label lying flat against the side of the rear part and/or front part in the tightened state thereof.

For identification purposes, the hygiene product according to the invention is provided with a label. The label is only connected at the two lateral edges, which extend perpendicularly to the upper edge, to the rear part and/or front part, on which the label is arranged. In addition, the label is connected to the rear part and/or front part such that it lies flat against the side of the rear part and/or front part in the tightened state thereof. For this purpose, the points at which the two lateral edges of the label are connected to the rear part and/or front part are at a distance from one another in the tightened state of the rear part and/or front part that corresponds to the distance between the two lateral edges of the label when spread out flat. As a result, the marking is also legible when the upper edge of the hygiene product is bunched up. That is to say, when the upper edge of the hygiene product is bunched up, the label is not bunched up since it is only connected at the two lateral edges thereof to the rear part and/or the front part. If the label is merely flexible and not elastic, it can bulge inward such that it is can still be read easily. If the label itself is elastic, it can contract such that the imprint on the flat side is still easy to read. This makes the hygiene product easier to handle, since it does not have to be tightened at the upper edge in order to check the marking. When the upper edge of the hygiene product is tightened, the label is in any case easy to read, since it lies flat against the inside of the rear part and/or front part. In addition, arranging the marking on a label produces a similarity with conventional underwear, which are preferably also marked by means of labels. However, unlike conventional underwear, the label is not connected to the underpants in a planar manner or peripherally around the outer edge, but rather only at the two lateral edges, in order to ensure legibility in the bunched-up state.

According to an embodiment of the invention, the label is connected at the two lateral edges thereof to the rear part and/or the front part along the entire or substantially the entire length of said two lateral edges. According to another embodiment, the label is connected at the two lateral edges thereof to the rear part and/or the front part in an uninterrupted manner. According to another embodiment, the label is connected at the two lateral edges thereof to the rear part and/or the front part in each case along broken lines and/or at points.

According to another embodiment, the label is arranged on the inside of the rear part and/or front part. As a result, greater similarity with conventional underwear is achieved. For even greater similarity, the label is arranged on one side of the rear part.

By arranging the label in the region of the elastication means, it is arranged in the region in which the hygiene product is bunched up by means of the elastication means in the slack state. As a result, arrangement of the label at the same height as the elastication means and arrangement of the label in a region below or above the elastication means that is bunched up by means of said elastication means in the slack state are included.

According to another embodiment, the label is arranged below an elastic hip band that is arranged in the upper edge region of the rear part and/or front part and that forms the elastication means that cause the upper edge to bunch up when slack. The arrangement below the elastic hip band is advantageous for securing the label.

According to another embodiment, the label is manufactured from a textile material. As a result, the wearing comfort is improved and greater similarity with conventional underwear is achieved. For this purpose, according to another embodiment, the label is manufactured from a nonwoven fabric. The design as nonwoven fabric is also advantageous for printing on a marking.

According to another embodiment, the label is manufactured from a material that has no or substantially no elasticity. According to another embodiment, the label is manufactured from an elastic material that is applied stretched to the tightened rear part and/or front part such that it lies flat against the side of the rear part and/or front part in the bunched-up state thereof. In this embodiment, the label can be read easily regardless of whether the upper edge is tightened or bunched up, since it always lies flat against one side of the rear part and/or front part. As a result, greater similarity with conventional underwear is achieved. According to another embodiment, the label has the same or substantially the same elasticity as the hygiene product in the region of the elastication means, in which the label is arranged.

During production, the label is preferably connected to the supporting material when said material is tightened. This may be done before or after the supporting material has been provided with the elastication means. Preferably, the elastication means is elastically stretched when applied to the supporting material and the supporting material is tightened and, in this state, the label is applied flat or elastically stretched to the supporting material and connected thereto at the lateral edges.

According to another embodiment, the label is printed. By printing on the marking, manufacture is particularly cost-effective. In principle, however, it is also possible to provide the label with a marking in another way, for example by means of stitching. The invention also includes embodiments in which the label bears no marking, such that it can be provided with a marking by hand before use, for example.

According to another embodiment, the label is printed with one color or a plurality of colors.

According to another embodiment, the label is printed by means of at least one inkjet print head. This is advantageous for fast and cost-effective manufacture.

According to another embodiment, the label is connected at the lateral edges to the rear part and/or front part by means of ultrasound or hot-melt adhesive. This is also advantageous for fast and/or cost-effective manufacture.

According to another embodiment, the marking comprises one or more of the following items of information: size, absorption strength, style, fit, gender of wearer, directions for use, manufacturer, brand, logo or other image.

The invention will be explained in greater detail below based on the following descriptions of exemplary embodiments.

### BRIEF DESCRIPTION OF THE DRAWINGS

FIG. 1 shows a hygiene product in underpants form spread out flat in a plan view of the inside;

FIG. 2 shows the same hygiene product in the tightened state in a perspective view obliquely from above;

FIG. 3 shows the same hygiene product in the same state in a section along the line II-II from FIG. 1;

FIG. 4 shows the same hygiene product having a label made of elastic material in the bunched-up state in the same section as in FIG. 3;

FIG. 5 shows the same hygiene product having a label made of non-elastic, flexible material in the bunched-up state in the same section as in FIG. 3; and

FIG. 6 shows the same hygiene product as in FIG. 5 in the bunched-up state in an enlarged plan view of the label and the inside of the rear part.

### DETAILED DESCRIPTION OF THE PREFERRED EMBODIMENTS

In the present application, the terms “upper” and “lower” as well as “front part” and rear part” relate to the position of use of the hygiene product on the body of a user standing upright.

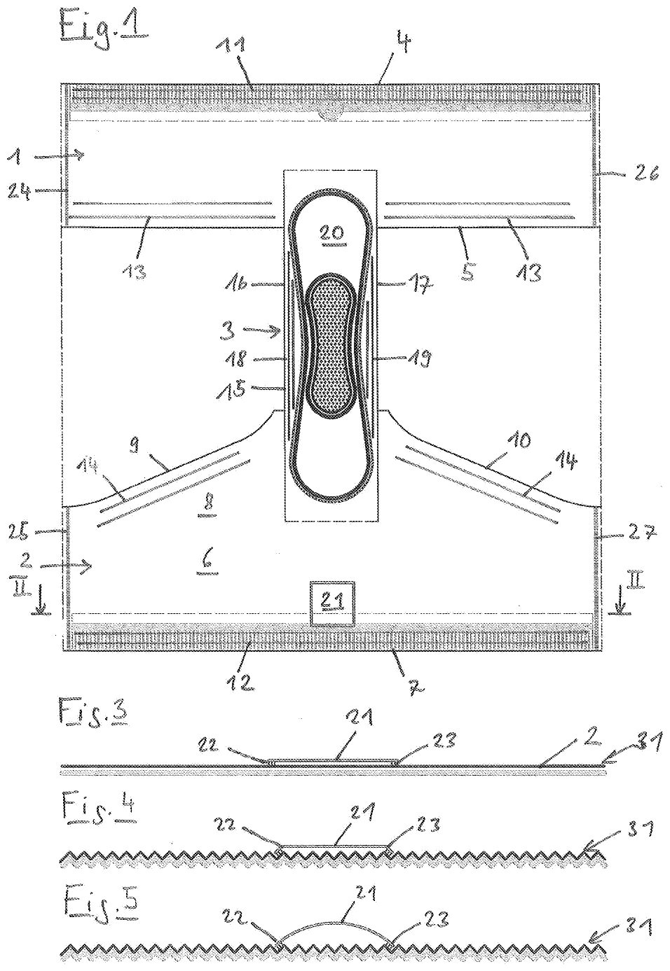

According to FIG. 1, the hygiene product comprises a front part 1, a rear part 2 and a crotch region 3 that connects the front part 2 to the rear part 3.

The front part 1 is rectangular with an upper edge 4 and a lower edge 5 that extends in parallel therewith.

The rear part 2 has a rectangular upper portion 6 with an upper edge 7 and a substantially trapezoidal lower portion 8 with lower edges 9, 10, which are arranged at an acute angle to the upper edge 7 and the distance of which from the lower edge increases as they approach the crotch region 3.

The front part 1 and the rear part 2 are in each case laminates having a protective film on the outside and a nonwoven fabric on the inside. First elastication means 11, 12 are arranged next to the upper edges 4, 7 and in parallel therewith. Second elastication means 13, 14 are arranged next to the lower edges 5, 9, 10 and in parallel therewith.

The crotch region 3 comprises a rectangular support strip 15, which is also a laminate having a protective film on the outside and a nonwoven fabric on the inside. Two additional elastication means 18, 19 are arranged in parallel with the outer edges 16, 17 of the support strip 15 between the protective film and the nonwoven fabric.

The first elastication means 11, 12 and the second elastication means 13, 14, 18, 19 are in each case formed of threads made of an elastomeric material, for example threads made of spandex (Lycra®). The threads extend in parallel with the respective edges, next to which the relevant elastication means are arranged.

An absorbent core 20 for absorbing liquid from a wearer is arranged on the inside of the support strip 15, which absorbent core is dumbbell-shaped in the example in the plan view. The absorbent core 20 comprises an absorbent material that contains cellulose and that also contains a superabsorbent. The material containing cellulose is preferably covered by an acquisition and distribution layer on the inside and wrapped in at least one layer of nonwoven fabric, which preferably only covers the lateral edge regions of the acquisition layer.

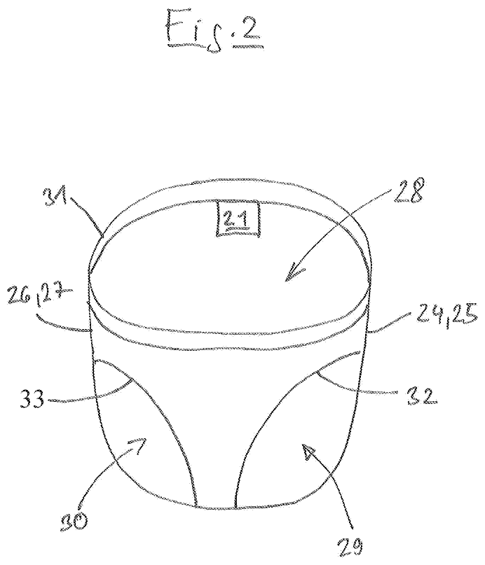

A label 21 is secured on the inside of the rear part 3 next to the first elastication means 12. According to FIG. 2, the label 21 is secured to the rear part 2 exclusively at the two lateral edges 22, 23, which extend perpendicularly to the upper edge 7 of the rear part 3. The securing is carried out, for example, by means of hot-melt adhesive or ultrasound.

The label 21 is provided on the inside with an imprint, which for example comprises information about the size, strength and style as well as the gender of the intended user.

FIG. 1 shows a semi-finished product of the hygiene product. In order to complete the hygiene product, the adjoining outer edges 24 and 25 as well as 26 and 27 of the front part 1 and rear part 2 have to be connected to one another. The connection is carried out for example by laying the adjoining outer edges 24 and 25 as well as 26 and 27 of the front part 1 and rear part 2 one on top of the other and fusing said edges together by means of ultrasound.

According to FIG. 2, in the finished hygiene product, the upper edges 4, 7 of the front part 1 and rear part 2 define a hip opening 28 and the lower edges 5, 9, 10 of the front part 1 and rear part 2 and the relevant outer edge 16, 17 of the support strip 15 connecting same define leg openings 29, 30. Taken together, the upper edges 4, 7 of the front part 1 and rear part 2 thus form the upper edge 31 of the hygiene product that defines the hip opening 28 and the lower edges 5, 9, 10 of the front part 1 and rear part 2 as well as the outer edges 16, 17 of the support strip connecting same form lateral edges 32, 33 of the hygiene product that define the leg openings 29, 30.

When the rear part 2 is tightened, the label 21 is also tightened and lies flat against the inside of the rear part 2. Consequently, the information on the label 21 is easy to read. This is demonstrated in FIG. 3.

When the hygiene product is slack, the first elastication means 11, 12, in particular, contract such that the hygiene product is bunched up, in particular at the upper edge 31.

In the exemplary embodiment from FIG. 4, the label 21 consists of an elastic material that has been elastically stretched onto the hygiene product in the tightened state according to FIG. 1. Consequently, when the upper edge 31 of the hygiene product is bunched up, the label 21 remains tight and the imprint is still easy to read.

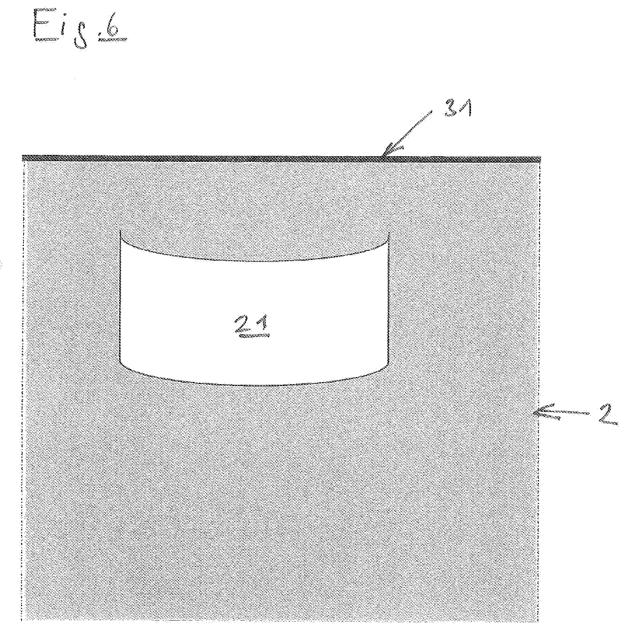

In the exemplary embodiment from FIG. 5, the label 21 consists of a flexible, but non-elastic or substantially non-elastic, material. The label 21 is applied so as to lie flat against the inside of the hygiene product according to FIG. 1 in the tightened state thereof and is fastened thereto. Consequently, when the hygiene product is bunched up, said label bulges out inward slightly, as shown in FIGS. 4 and 5. Nevertheless, the imprint on the label is easy to read.

### LIST OF REFERENCE SIGNS

1 Front part
2 Rear part
3 Crotch region
4 Upper edge
5 Lower edge
6 Upper portion
7 Upper edge
8 Lower portion
9, 10 Lower edge
11, 12 First elastication means
13, 14 Second elastication means
15 Support strip
16, 17 Outer edge
18, 19 Second elastication means
20 Absorbent core
21 Label
22, 23 Lateral edge
24 to 27 Outer edge
28 Hip opening
29, 30 Leg opening
31 Upper edge
32, 33 Lateral edge

I claim:

1. A hygiene product in underpants form comprising: a front part (1) and a rear part (2) as well as a crotch region (3) connecting the front part (1) to the rear part (2), the front part (1) and rear part (2) defining a hip opening (28) at the upper edge (30) and the front part (1), rear part (2) and crotch region (3) defining leg openings (29, 30) at two lateral edges (32, 33), an absorbent core (20) being arranged in the crotch region (3) and the front part (1) and rear part (2) comprising elastication means (11, 12) that extend in parallel with the upper edge (30) and that cause the upper edge to bunch up when slack, wherein a label (21) is arranged in the region of the elastication means (11, 12) on at least one side of the rear part (2) and/or front part (1), which label is only connected at the two lateral edges thereof (22, 23), which extend perpendicularly to the upper edge (30), to the rear part (2) and/or front part (1), the label (21) lying flat against the side of the rear part (2) and/or front part (1) in the tightened state thereof.

2. The hygiene product according to claim 1, wherein the label (21) is arranged on the inside of the rear part (2) and/or front part (1).

3. The hygiene product according to claim 1, wherein the label (21) is arranged on one side of the rear part (2).

4. The hygiene product according to claim 1, wherein the label (21) is arranged below an elastic hip band arranged in the upper edge region of the rear part (2) and/or front part (1).

5. The hygiene product according to claim 1, wherein the label (21) is manufactured from a textile material.

6. The hygiene product according to claim 5, wherein the label (21) is manufactured from a nonwoven fabric.

7. The hygiene product according to claim 1, wherein the label (21) is manufactured from an elastic material that is applied stretched to the tightened rear part (2) and/or front part (1) such that it lies flat against the side of the rear part (2) and/or front part (1) in the bunched-up state thereof.

8. The hygiene product according to claim 1, wherein the label (21) is printed.

9. The hygiene product according to claim 8, wherein the label (21) is printed with one color or a plurality of colors.

10. The hygiene product according to claim 8, wherein the label (21) is printed by means of at least one inkjet print head.

11. The hygiene product according to claim 1, wherein the label (21) is connected at the lateral edges (22, 23) to the rear part and/or front part by means of ultrasound or holt-melt adhesive.

12. The hygiene product according to claim 1, wherein the label (21) comprises one or more of the following markings: size, absorption strength, style, fit, gender of wearer, directions for use, disposal instructions, manufacturer, brand, logo or other image.
